(19) United States
(12) Patent Application Publication
(10) Pub. No.: US 2024/0261265 A1
(43) Pub. Date: Aug. 8, 2024

(54) TINOSTAMUSTINE FOR USE IN TREATING OVARIAN CANCER

(71) Applicant: Purdue Pharma L.P., Stamford, CT (US)

(72) Inventor: Thomas Jorg Mehrling, Riehen (CH)

(73) Assignee: Purdue Pharma L.P., Stamford, CT (US)

(21) Appl. No.: 18/404,810

(22) Filed: Jan. 4, 2024

Related U.S. Application Data
(63) Continuation of application No. 16/621,893, filed on Dec. 12, 2019, now Pat. No. 11,896,583.

(30) Foreign Application Priority Data
Jun. 13, 2017 (GB) 1709405.3

(51) Int. Cl. A61K 31/4184; A61K 31/337; A61K 33/243; A61P 35/00

(52) U.S. Cl. CPC A61K 31/4184; A61K 31/337; A61K 33/243; A61P 35/00

(57) ABSTRACT

There is provided tinostamustine or a pharmaceutically acceptable salt thereof for use in the treatment of ovarian cancer in a patient in need thereof.

### REFERENCES TO RELATED APPLICATIONS

This application is a continuation of U.S. patent application Ser. No. 16/621,893, filed on Dec. 12, 2019, which is a U.S. National Stage application filed under 35 U.S.C. 371 of International Patent Application No. PCT/EP2018/065669, filed on Jun. 13, 2018, which claims priority to United Kingdom Patent Application No. 1709405.3, filed on Jun. 13, 2017. The entire contents of each of the aforementioned applications, including drawings, are incorporated herein by reference.

### TECHNICAL FIELD

The present invention relates to methods of treating cancer, particularly ovarian cancer.

### BACKGROUND TO THE INVENTION

Cancer is one of the most life threatening diseases. Cancer is a condition in which cells in a part of the body experience out-of-control growth. According to latest data from American Cancer Society, it is estimated there will be 1.69 million new cases of cancer in USA in 2017. Cancer is the second leading cause of death in the United States (second only to heart disease) and will claim more than 601,000 lives in 2017. In fact, it is estimated the average lifetime risk of developing cancer is 40.8% for American males and 37.5% for American women. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumours located at certain sites, for example, in the breast, colon, and skin, it cannot be used in the treatment of tumours located in other areas, such as the backbone, nor in the treatment of disseminated hematological cancers including cancers of the blood and blood-forming tissues (such as the bone marrow). Such cancers include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in chemotherapy is the development of drug resistance by the cancer cells, a serious problem that may lead to recurrence of disease or even death. Thus, more effective cancer treatments are needed.

Solid tumours are an abnormal mass of tissue that usually does not contain cysts or liquid areas. Solid tumours may be benign (not cancer), or malignant (cancer). Different types of solid tumours are named for the type of cells that form them. Examples of solid tumours are carcinomas and sarcomas. The four most common cancers occurring worldwide are all solid tumours, namely lung, breast, bowel and prostate cancer. These four solid tumour cancers account for around 4 in 10 of all cancers diagnosed worldwide.

According to the American Cancer Society, estimates for ovarian cancer in the United States for 2017 are that about 22,440 women will receive a new diagnosis of ovarian cancer and about 14,080 women will die from ovarian cancer.

Ovarian cancer ranks fifth in cancer deaths among women, accounting for more deaths than any other cancer of the female reproductive system. A woman's risk of getting ovarian cancer during her lifetime is about 1 in 75. Her lifetime chance of dying from ovarian cancer is about 1 in 100.

Ovarian cancer mainly develops in older women. About half of the women who are diagnosed with ovarian cancer are 63 years or older. It is more common in white women than African-American women.

Given the prevalence of ovarian cancer, there is therefore a need for new effective chemotherapeutic treatments.

In WO-A-2010/085377, the compound of formula I below is disclosed. It is a first-in-class dual-functional alkylating-HDACi fusion molecule which potently inhibits HDAC-regulated pathways.

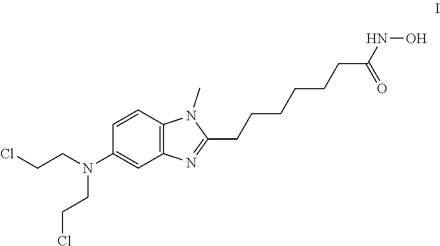 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM). The compound of formula I has an INN of tinostamustine and is also known in the art as EDO-S101. It is an AK-DAC (a first-in-class alkylating deacetylase molecule) that, in preclinical studies, has been shown to simultaneously improve access to the DNA strands within cancer cells, break them and block damage repair.

### SUMMARY OF THE INVENTION

In a first aspect of the present invention there is provided tinostamustine or a pharmaceutically acceptable salt thereof for use in the treatment of ovarian cancer in a patient in need thereof.

It has surprisingly been discovered that tinostamustine or a pharmaceutically acceptable salt thereof is particularly effective in the treatment of ovarian cancer, with activity data showing strong sensitivity to this compound. Thus, the need for a new and effective treatment of ovarian cancer is met by the present invention.

In a further aspect of the present invention there is provided the use of tinostamustine or a pharmaceutically acceptable salt thereof for the manufacture of a medicament for the treatment of ovarian cancer.

In a further aspect of the present invention there is provided a method of treating ovarian cancer in a patient in need thereof comprising administering to said patient an effective amount of tinostamustine or a pharmaceutically acceptable salt thereof.

In a further aspect of the present invention there is provided a kit comprising tinostamustine or a pharmaceutically acceptable salt thereof together with instructions for treating ovarian cancer.

In a further aspect, tinostamustine or a pharmaceutically acceptable salt thereof may be administered in combination with one or more further agent(s). The further agent may be carboplatin. The further agent may be paclitaxel. The further agents may be both carboplatin and paclitaxel.

When used in combination, tinostamustine or a pharmaceutically acceptable salt thereof and further agent(s) can be administered concurrently, sequentially or separately. In an embodiment, the agents are administered concurrently. In a further embodiment, the agents are administered sequentially. In a yet further embodiment, the agents are administered separately.

In a further aspect, there is provided a combination comprising tinostamustine or a pharmaceutically acceptable salt thereof and a further agent(s) selected from: carboplatin and/or paclitaxel.

Combinations of tinostamustine or a pharmaceutically acceptable salt thereof and a further agent(s) selected from carboplatin and/or paclitaxel have been found to be particularly effective in the treatment of cancers, such as ovarian cancer, such that they are highly promising in efforts to address the problems of finding more effective treatments of cancer.

Tinostamustine or a pharmaceutically acceptable salt thereof, and further agent(s) can be administered concurrently, sequentially or separately. In an embodiment, the agents are administered concurrently. In a further embodiment, the agents are administered sequentially. In a yet further embodiment, the agents are administered separately.

In a further aspect, there is provided a pharmaceutical composition comprising a pharmaceutically acceptable carrier and a combination according to the present invention.

In a further aspect, there is provided a kit comprising a combination according to the present invention and optionally instructions for treating a patient.

In a further aspect, there is provided a combination according to the present invention for use as a medicament.

In a further aspect, there is provided a combination according to the present invention for use in the treatment of cancer.

In a further aspect, there is provided the use of a combination according to the present invention for the manufacture of a medicament for the treatment of cancer.

In a further aspect, there is provided a method of treating cancer in a patient in need thereof comprising administering a combination according to the present invention.

The following features apply to all aspects of the invention.

The cancer may be ovarian cancer.

The ovarian cancer may be from the omentum.

The ovarian cancer may be a primary cancer.

The ovarian cancer may be relapsed and/or refractory.

The ovarian cancer may be platinum resistant.

The ovarian cancer may be BRCA-1/2 wildtype.

The ovarian cancer may be localized.

The ovarian cancer may be metastatic.

The ovarian cancer may be advanced.

The ovarian cancer may have progressed after at least one line of standard therapy.

Tinostamustine may be administered as a monotherapy.

One or more further agent(s) may be administered in combination with tinostamustine or a pharmaceutically acceptable salt thereof.

The combination may be tinostamustine or a pharmaceutically acceptable salt thereof and a further agent or agents.

The further agent may be carboplatin.

The further agent may be paclitaxel.

The further agents may be carboplatin and paclitaxel.

### DESCRIPTION OF THE DRAWINGS

FIG. 1 shows the results of the feasibility study using samples from primary ovarian cancer patient #5284.

FIG. 2 shows the results of an identical feasibility study using samples from primary ovarian cancer patient #5285.

FIG. 3 shows the activity of EDO-S101 (at 5 μM) as a monotherapy in the ovarian cancer spheroid model (n=8).

FIG. 4 shows the activity of EDO-S101 as a combination therapy in the ovarian cancer spheroid model (n=8).

FIG. 5 compares EDO-S101+carboplatin and carboplatin+paclitaxel in platinum sensitive and platinum resistant models.

FIG. 6 provides treatment results evaluated with the ATP assay after 72 h using samples from primary ovarian cancer patient #5295.

FIG. 7 provides treatment results evaluated with the ATP assay after 72 h using samples from primary ovarian cancer patient #5296.

FIG. 8 provides treatment results evaluated with the ATP assay after 72 h using samples from primary ovarian cancer patient #5310 and compares EDO-S101 alone and in combination with carboplatin or paclitaxel in a tumour material collected from patient operations and taken from the ovary and from the omentum.

FIG. 9 provides treatment results evaluated with the ATP assay after 72 h using samples from primary ovarian cancer patient #5311.

FIG. 10 provides treatment results evaluated with the ATP assay after 72 h using samples from primary ovarian cancer patient #5329.

### DETAILED DESCRIPTION OF THE INVENTION

In the present application, a number of general terms and phrases are used, which should be interpreted as follows.

The compound of formula I has an INN of tinostamustine and is also known in the art as EDO-S101. The IUPAC name is 7-(5-(bis(2-chloroethyl)amino)-1-methyl-1H-benzo[d]imidazol-2-yl)-N-hydroxyheptanamide.

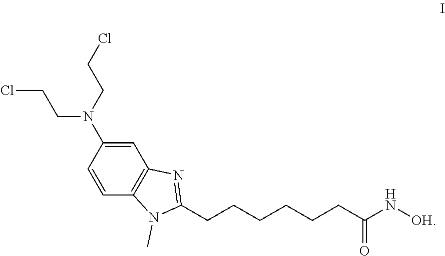 [SMILES: Cn1c(CCCCCCC(=O)NO)nc2cc(N(CCCl)CCCl)ccc21]

“Patient” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In the present invention, the pharmaceutically acceptable salt of tinostamustine may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate salt.

It has surprisingly been found that tinostamustine or a pharmaceutically acceptable salt thereof shows surprising efficacy in solid tumours. In particular, it has been found that tinostamustine or a pharmaceutically acceptable salt thereof is useful in the treatment of ovarian cancer.

According to the Ovarian Cancer Research Fund Alliance, ovarian cancer is the number one cause of gynecological cancer deaths, and is the 5th leading cause of cancer related deaths in women and is the 11th most common cancer in women. The five year survival rate of women diagnosed with ovarian cancer between 2007 and 2013 was 47% according to National Cancer Institute (US) statistics.

Three types of ovarian tumour are known to exist: carcinomas (epithelial), germ cell and sex-cord stromal. The most common type of ovarian cancer is ovarian carcinoma, which represents approximately 90% of all ovarian cancer occurrences according to Ovarian Cancer Action UK, and occurs most commonly in women between 40 and 60 years old. Approximately 10% of instances of ovarian cancer can be linked to inherited genetic mutations. Women who inherit such genetic mutations are up to 50% more likely to develop the disease than women without the mutations. Many of the most aggressive and mutagenic ovarian cancers have mutations in genes implicated in the DNA damage repair pathways; for examples mutations in genes such as BRCA-1, BRCA-2 and p53. In approximately 50% of highly aggressive ovarian cancers, normal DNA damage repair pathways are consequently dysfunctional. Other common genetic mutations found in ovarian cancer include mutations in NF1 which encodes transcription factors, and CDK12 which regulates the cell cycle.

Current standards of therapy for ovarian cancer include surgery, radiotherapy and chemotherapy. Chemotherapeutic agents include paxlitaxtel, carboplatin, cisplatin, topotecan, doxorubicin and gebcitabine. Many aggressive ovarian cancers are platinum-resistant, for which alternative drugs such as cyclophosphamides may be used.

Given the prevalence of ovarian cancer and resistance to current therapies, there is a need for new effective chemotherapeutic treatments.

The therapeutically effective amount of tinostamustine or a pharmaceutically acceptable salt administered to the patient is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of tinostamustine or a pharmaceutically acceptable salt thereof according to the present invention is believed to be one wherein tinostamustine or a pharmaceutically acceptable salt thereof is included at a dosage range of from 0.3 mg/m² to 300 mg/m² body surface area of the patient or from 60 mg/m² to 150 mg/m² body surface area of the patient. In a preferred embodiment, the dosage range is from 80 to 100 mg/m² body surface area of the patient.

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

“Primary cancer” is the original, or first, tumour in the body. Cancer cells from a primary tumor may spread to other parts of the body and form new, or secondary, tumors. This is called metastasis.

“Metastatic Cancer”. Cancer has the ability to spread within the body. Cancer cells can spread locally by moving into nearby normal tissue. Cancer can also spread regionally, to nearby lymph nodes, tissues, or organs. Cancer can therefore spread to distant parts of the body. When this happens, it is called metastatic cancer (also known as stage IV cancer), and the process by which cancer cells spread to other parts of the body is called metastasis. Thus, in metastasis, cancer cells break away from where they first formed (primary cancer), travel through the blood or lymph system, and form new tumours (metastatic tumours) in other parts of the body.

Metastatic cancer cells have features like that of the primary cancer and not like the cells in the place where the cancer is found. This enables doctors to tell whether a cancer is metastatic. Metastatic cancers are given the same name as the primary cancer. For example, breast cancer that has spread to the lung is called metastatic breast cancer, not lung cancer. It is treated as stage IV breast cancer, not as lung cancer.

Metastatic ovarian cancer refers to an ovarian cancer that has metastasised to a new location in the body. The cancer is treated as a stage IV ovarian cancer.

“Advanced Cancer” is a cancer that is not curable but responds to treatment. Disease directed therapy is still very important because it prolongs life. For terminal cancer, therapy cannot prolong survival significantly due to the progressive nature of the disease and palliative care is the main treatment option.

“Monotherapy” according to the present invention means tinostamustine or a pharmaceutically acceptable salt thereof is administered alone as a single agent. i.e. it is not administered in combination with a further drug or drugs. Examples of such drugs include (but are not limited to) proteasome inhibitors, glucocorticoids and/or tyrosine kinase inhibitors. However, a tinostamustine monotherapy may include radiotherapy.

Suitable examples of the administration form of tinostamustine or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques. Preferably, tinostamustine or a pharmaceutically acceptable salt thereof is administered parenterally, and most preferably intravenously.

“BRCA-1/2 wildtype”. BRCA-1 is a gene found on chromosome 17. BRCA-2 is a gene found on chromosome 13. BRCA-1 and BRCA-2 are human genes that produce tumour suppressor proteins. These proteins help repair damaged DNA and, therefore, play a role in ensuring the stability of each cell's genetic material. When either of these genes is mutated, or altered, such that its protein product is not made or does not function correctly, DNA damage may not be repaired properly. As a result, cells are more likely to develop additional genetic alterations that can lead to cancer. Specific inherited mutations in BRCA-1 and BRCA-2 most notably increase the risk of female breast and ovarian cancers, but they have also been associated with increased risks of several additional types of cancer. People who have inherited mutations in BRCA-1 and BRCA-2 tend to develop breast and ovarian cancers at younger ages than people who do not have these mutations.

A harmful BRCA-1 or BRCA-2 mutation can be inherited from a person's mother or father. Each child of a parent who carries a mutation in one of these genes has a 50% chance (or 1 chance in 2) of inheriting the mutation. The effects of mutations in BRCA-1 and BRCA-2 are seen even when a person's second copy of the gene is normal.

In contrast, a person who has a BRCA-1 and/or BRCA-2 gene which is not mutated has a ‘wildtype’ i.e. non-mutated gene. BRCA-1/2 wildtype means a patient who has non-mutated BRCA-1 and/or BRCA-2 genes. In an embodiment, BRCA-1/2 wildtype patients do not have a mutated BRCA-1 or BRCA-2 gene. In a further embodiment, BRCA-1/2 wildtype patients do not have a mutated BRCA-1 and BRCA-2 genes.

A woman's lifetime risk of developing breast and/or ovarian cancer is greatly increased if she inherits a harmful mutation in BRCA-1 or BRCA-2. About 12% of women in the general population will develop breast cancer sometime during their lives. By contrast, a recent large study estimated that about 72% of women who inherit a harmful BRCA-1 mutation and about 69% of women who inherit a harmful BRCA-2 mutation will develop breast cancer by the age of 80. Like women from the general population, those with harmful BRCA-1 or BRCA-2 mutations also have a high risk of developing a new primary cancer in the opposite (contralateral) breast in the years following a breast cancer diagnosis. It has been estimated that, by 20 years after a first breast cancer diagnosis, about 40% of women who inherit a harmful BRCA-1 mutation and about 26% of women who inherit a harmful BRCA-2 mutation will develop cancer in their other breast. About 1.3% of women in the general population will develop ovarian cancer sometime during their lives. By contrast, it is estimated that about 44% of women who inherit a harmful BRCA-1 mutation and about 17% of women who inherit a harmful BRCA-2 mutation will develop ovarian cancer by the age of 80. As such, patients having mutated BRCA-1 and/or BRCA-2 genes carry an increased risk of getting cancer.

At the same time, the presence of mutations on the BRCA-1 and/or BRCA-2 genes limit DNA repair pathways of BRCA-1/2 mutant tumours, making these tumours more susceptible to certain therapies including DNA damaging agents like platinum agents. Thus, patients who are BRCA-1/2 wildtype may have worse prognosis and outcomes.

Preferably, tinostamustine or a pharmaceutically acceptable salt thereof is administered intravenously to the patient in need thereof at a dosage level to the patient in need thereof of from 0.3 mg/m² to 300 mg/m² body surface area of the patient.

Preferably, tinostamustine or a pharmaceutically acceptable salt thereof is administered intravenously to the patient in need thereof at a dosage level to the patient in need thereof of from 60 mg/m² to 150 mg/m² body surface area of the patient.

Preferably, tinostamustine or a pharmaceutically acceptable salt thereof is administered intravenously to the patient in need thereof at a dosage level to the patient in need thereof of from 80 mg/m² to 100 mg/m² body surface area of the patient.

It has been found that in embodiments of the present invention, tinostamustine or a pharmaceutically acceptable salt thereof or medicament comprising the same may preferably be administered to a patient in need thereof on days 1, 8 and 15 of a 28 day treatment cycle or on days 1 and 15 of a 28 day treatment cycle.

Preferably, tinostamustine or a pharmaceutically acceptable salt thereof is administered on days 1 and 15 of a 28 day treatment cycle.

It has been found that in embodiments of the present invention, tinostamustine or a pharmaceutically acceptable salt thereof or medicament comprising the same may preferably be administered to a patient in need thereof over an infusion time of 60 minutes; or an infusion time of 45 minutes; or an infusion time of 30 minutes.

Preferably, tinostamustine or a pharmaceutically acceptable salt thereof is administered over an infusion time of 60 minutes.

In a preferred embodiment, tinostamustine or a pharmaceutically acceptable salt is administered to the patient in need thereof at a dosage level of from 80 mg/m² to 100 mg/m² body surface area of the patient, on days 1 and 15 of a 28 day treatment cycle, over an infusion time of 60 minutes.

In embodiments of the present invention, there is provided a kit comprising tinostamustine or a pharmaceutically acceptable salt thereof or medicament comprising the same together with instructions.

The instructions may advise administering tinostamustine or a pharmaceutically acceptable salt thereof according to variables such as the state of the solid tumours being treated; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

In a further embodiment of the present invention, the patient in need of said treatment is given radiotherapy with (including prior to, during or after) treatment of the solid tumour(s) with tinostamustine or a pharmaceutically acceptable salt thereof. In embodiments of the present invention, the patient is treated with tinostamustine or a pharmaceutically acceptable salt thereof and radiotherapy. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

In a further embodiment of the present invention, the patient in need of said treatment is given radiotherapy prior to or after treatment of the solid tumours with tinostamustine or a pharmaceutically acceptable salt thereof. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

When intended for oral administration, tinostamustine or a pharmaceutically acceptable salt thereof or medicament comprising the same may be in solid or liquid form, where semi-solid, semi-liquid, suspension and gel forms are included within the forms considered herein as either solid or liquid.

Tinostamustine or a pharmaceutically acceptable salt thereof or medicament comprising the same can be prepared for administration using methodology well known in the pharmaceutical art. Examples of suitable pharmaceutical formulations and carriers are described in “Remington's Pharmaceutical Sciences” by E. W. Martin.

As a solid composition for oral administration, tinostamustine or a pharmaceutically acceptable salt thereof can be formulated into a powder, granule, compressed tablet, pill, capsule, chewing gum, wafer or the like form. Such a solid composition typically contains one or more inert diluents or carriers. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When tinostamustine or a pharmaceutically acceptable salt thereof compositions is in the form of a capsule (e.g. a gelatin capsule), it can contain, in addition to materials of the above type, a liquid carrier such as polyethylene glycol, cyclodextrin or a fatty oil.

Tinostamustine or a pharmaceutically acceptable salt thereof compositions can be in the form of a liquid, e.g. an elixir, syrup, solution, emulsion or suspension. The liquid can be useful for oral administration or for delivery by injection When intended for oral administration, tinostamustine or a pharmaceutically acceptable salt thereof compositions can comprise one or more of a sweetening agent, preservatives, dye/colorant and flavor enhancer. In tinostamustine or a pharmaceutically acceptable salt thereof compositions for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, tinostamustine or a pharmaceutically acceptable salt thereof or medicament comprising the same is administered intravenously.

Liquid forms of tinostamustine or a pharmaceutically acceptable salt thereof or medicament comprising the same, may be solutions, suspensions or other like form, and can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

Tinostamustine or a pharmaceutically acceptable salt thereof or medicament comprising the same can be administered by any convenient route, for example by infusion or bolus injection, by absorption through epithelial or mucocutaneous linings, and preferably by bolus.

Examples of compositions comprising tinostamustine or a pharmaceutically acceptable salt thereof are disclosed in WO2013/040286.

The present invention encompasses combinations of tinostamustine or a pharmaceutically acceptable salt and carboplatin and/or paclitaxel.

In one embodiment of the combination of the present invention, tinostamustine or a pharmaceutically acceptable salt thereof and a further agent(s) are adapted for administration concurrently, sequentially or separately. Preferably, tinostamustine or a pharmaceutically acceptable salt thereof and the further agent(s) are adapted for administration concurrently.

In one embodiment of the combination of the present invention, the combination comprises tinostamustine or a pharmaceutically acceptable salt thereof; and carboplatin.

In one embodiment of the combination of the present invention, the combination comprises tinostamustine or a pharmaceutically acceptable salt thereof; and paclitaxel.

In one embodiment of the combination of the present invention, the combination comprises tinostamustine or a pharmaceutically acceptable salt thereof; and carboplatin and paclitaxel.

The molar ratio of carboplatin to tinostamustine or a pharmaceutically acceptable salt thereof in the combination of the present invention is typically from 1:2000 to 2000:1. Preferably, the molar ratio of carboplatin to tinostamustine or a pharmaceutically acceptable salt thereof in said combination is from 1:2000 to 1:100, more preferably the molar ratio of carboplatin to tinostamustine or a pharmaceutically acceptable salt thereof in said combination is from 1:1000 to 1:500, and most preferably it is from 1:900 to 1:500, e.g. 1:900, 1:800, 1:700, 1:600 or 1:500.

The molar ratio of paclitaxel to the tinostamustine or a pharmaceutically acceptable salt thereof in the combination of the present invention is typically from 1:2000 to 2000:1. Preferably, the molar ratio of paclitaxel to tinostamustine or a pharmaceutically acceptable salt thereof in said combination is from 1:2000 to 1:100, more preferably the molar ratio of paclitaxel to tinostamustine or a pharmaceutically acceptable salt thereof in said combination is from 1:1000 to 1:500, and most preferably it is from 1:900 to 1:500, e.g. 1:900, 1:800, 1:700, 1:600 or 1:500.

It has been surprisingly found that combinations comprising tinostamustine or a pharmaceutically acceptable salt thereof and a further agent are synergistic combinations. In other words, the potency of the combinations has been measured with the Calcusyn software (biosoft, Ferguson, MO, USA), which is based on the Chou Talay method (Chou et al., Adv. Enzyme Regul., 22, 27-55 (1984)), that calculates a combination index (CI) with the following interpretation:

$$C I 1 > 1 : antagonist effect , \\ C I = 1 : additive effect and C I < 1 synergistic effect .$$

The present invention may be further understood by consideration of the following non-limiting examples.

### EXAMPLES

In the following examples, tinostamustine is referred to as EDO-S101.

EDO-S101 may be prepared as described in Example 6 of WO-A-2010/085377.

### Example 1—Activity of EDO-S101 in an Ovarian Cancer Spheroid Model

The activity of EDO-S101 alone and in combination was evaluated in ovarian cancer cells lines using a spheroid model.

### EDO-S101 and Control Compounds

EDO-S101 was provided by EDO MundiPharma, and synthesised as described in Example 6 of WO-A-2010/085377.
Paclitaxel was purchased from Sigma
Carboplatin was purchased from Sigma
Vorinostat was purchased from Sigma

### Materials and Methods

Fresh primary ovarian cancer samples were obtained after informed consent of the individual patient was given. Patients were recruited following clinical diagnosis, and prior to chemotherapy commencing.

Heterotypic tumour spheroids were prepared from the individual primary ovarian cancer tissue. The tumour samples were mechanically and enzymatically digested using an enzyme cocktail (Roche, Penzberg Germany), cell viability determined using the trypan-blue exclusion test, and spheroids seeded at a cell density of 50,000 using a modified liquid overlay method. After 48 h in cell culture under standard conditions, successful spheroid formation was documented photographically before starting treatment.

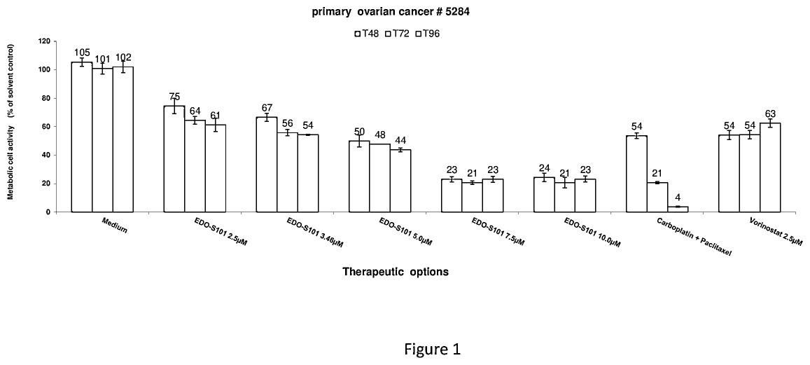

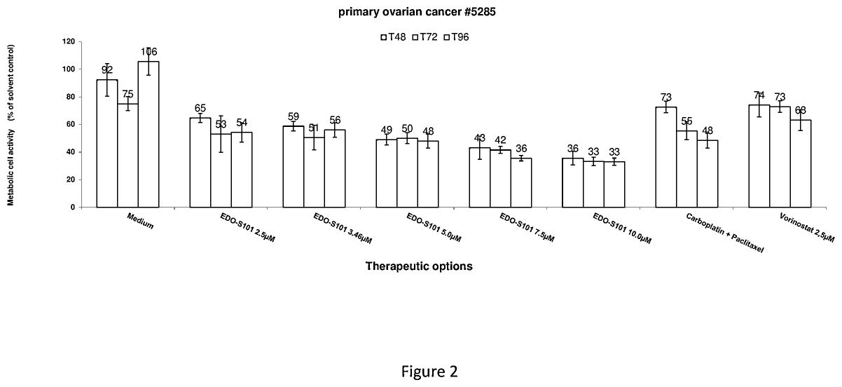

The spheroids were treated with EDO-S101 in five different concentrations 2.5 μM, 3.46 μM, 5 μM, 7.5 μM, 10 μM for three different time points (48 h, 72 h, 96 h) in an initial feasibility study. Each experiment was repeated six times, and both untreated (medium) control and two positive controls (carboplatin+paclitaxel and vorinostat) were used under the same culture conditions. Treatment efficacy, i.e. treatment-induced reduction of the cellular metabolic activity (cell viability) was analyzed using a standard ATP assay. The results of the experiments are shown in FIGS. 1-2.

Once the feasibility study was completed, tumour spheroids consisting of 50,000 cells were prepared from the individual primary ovarian cancer tissue as described above. After 48 h, spheroid formation was documented photographically before starting treatment. The spheroids were then treated (n=6) with EDO-S101 (5 μM) as single agent and in combination therapy with carboplatin (110 μM) and paclitaxel (1.79 μM) for 72 h. Treatment efficacy was evaluated using a standard ATP assay, details of which are provided below.

ATP assay test from Promega: CellTiter-Glo®. The CellTiter-Glo® Luminescent Cell Viability Assay is a homogeneous method to determine the number of viable cells in culture based on quantitation of the ATP present, which signals the presence of metabolically active cells. The CellTiter-Glo® Assay is designed for use with multiwell plate formats, making it ideal for automated high-throughput screening (HTS), cell proliferation and cytotoxicity assays. The homogeneous assay procedure involves adding a single reagent (CellTiter-Glo® Reagent) directly to cells cultured in serum-supplemented medium. Cell washing, removal of medium or multiple pipetting steps are not required.

The homogeneous “add-mix-measure” format results in cell lysis and generation of a luminescent signal proportional to the amount of ATP present. The amount of ATP is directly proportional to the number of cells present in culture, in agreement with previous reports. The CellTiter-Glo® Assay generates a “glow-type” luminescent signal, produced by the luciferase reaction, which has a half-life of greater than five hours. This extended half-life eliminates the need for reagent injectors and provides flexibility for continuous or batch-mode processing of multiple plates. The unique homogeneous format reduces pipetting errors that may be introduced during the multiple steps required by other ATP measurement methods.

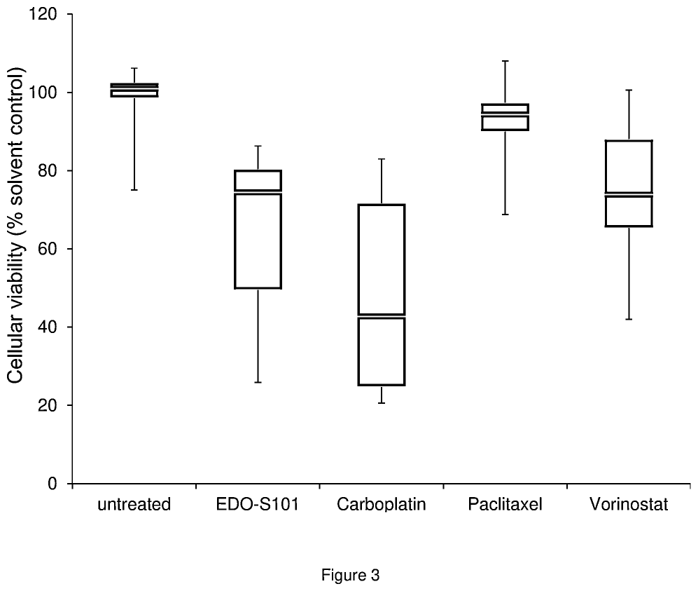

The results of the experiments are shown in FIGS. 3-6.

FIG. 1 shows the results of the feasibility study in an ovarian cancer sample. A reduction in cell viability was observed in spheroids treated with 2.5-10 μM EDO-S101, compared to the control untreated spheroids. At lower concentrations of EDO-S101, cell viability decreased in a time dependent manner from 48 h to 72 h and from 72 h to 96 h. For higher concentrations of EDO-S101, cell viability did not decrease in a time dependent manner. A reduction in cell viability to 50% was observed for spheroids treated with 5 μM of EDO-S101 after 48 h, whereas the higher concentrations of 7.5 and 10 μM achieved a reduction in cell viability of nearly 80% (compared to the untreated control). The reduction in cell viability observed in spheroids treated with 5, 7.5 and 10 μM EDO-S101 was greater than that in spheroids treated with vorinostat at all time points. Furthermore, the reduction in cell viability of spheroids treated with 5, 7.5 and 10 μM EDO-S101 was greater than that in spheroids treated with carboplatin+paclitaxel after 48 h and was comparable after 72 h.

FIG. 2 shows the results of an identical feasibility study in a different ovarian cancer sample. In this instance EDO-S101 achieves similar reduction in cell viability (though not as strong a reduction for the high concentrations) whereas the positive controls are less effective. In this second feasability study, EDO-S101 is better than both carboplatin+paclitaxel or vorinostat. These data suggest that this patient's ovarian cancer sample was possibly more drug resistant than the patient's sample used in example 1, and therefore that EDO-S101 may be more effective at overcoming said drug resistance.

FIG. 3 shows the activity of EDO-S101 (at 5 μM) as a monotherapy in the ovarian cancer spheroid model (n=8) compared with other monotherapies. It can be seen that paclitaxel is comparable to the negative control and is therefore largely ineffective in reducing cell viability. Treatment with EDO-S101 revealed a median reduction in cell viability comparable to vorinostat but with enhanced average potency across the experimental group. It can also be seen that the range in reduction of cell viability in spheroids treated with EDO-S101 was comparable to those treated with carboplatin.

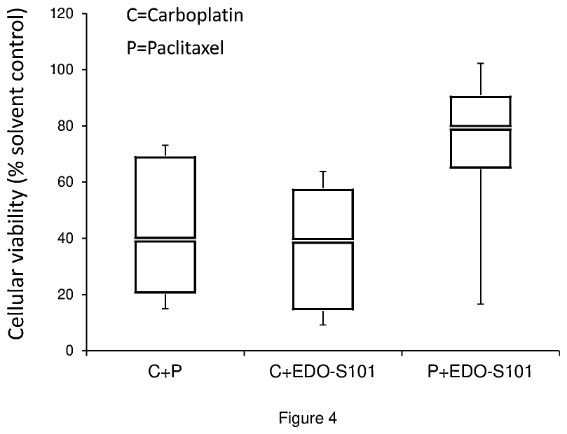

FIG. 4 shows the activity of EDO-S101 as a combination therapy in the ovarian cancer spheroid model (n=8). It can be seen that EDO-S101+carboplatin is more effective than EDO-S101+paclitaxel. It can also be seen that EDO-S101 shows a median reduction in cell viability comparable to carboplatin+paclitaxel, but with enhanced average potency across the experimental group. This suggests EDO-S101 may potentiate carboplatin in ovarian cancer.

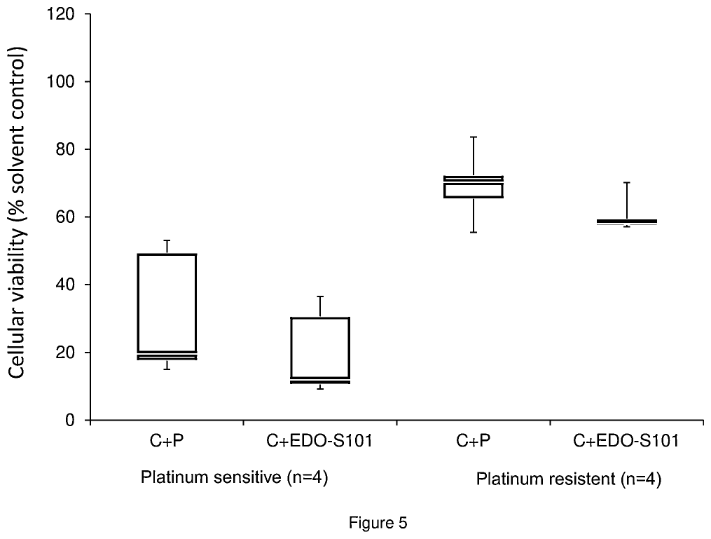

FIG. 5 compares EDO-S101+carboplatin and carboplatin+paclitaxel in platinum sensitive and platinum resistant models. EDO-S101+carboplatin is more effective than carboplatin+paclitaxel in both platinum sensitive and platinum resistant models, exhibiting reduced median cell viability in both cases, and a smaller range and enhanced average potency across the experimental group. In relation to platinum resistant ovarian cancer, EDO-S101+carboplatin reduced the median cell viability observed by approximately 10%. This suggests treatment of platinum resistant ovarian cancers with EDO-S101 may be beneficial compared to conventional combination therapies.

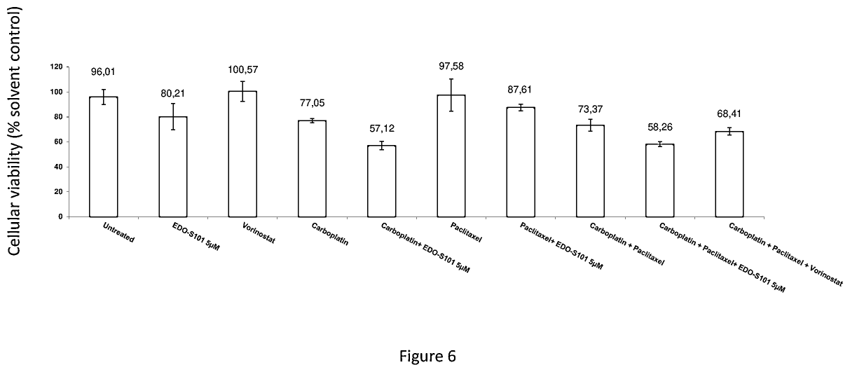

FIG. 6 provides treatment results evaluated with the ATP assay after 72 h using samples from primary ovarian cancer patient #5295. EDO-S101 as a monotherapy shows a reduction in metabolic activity compared to the negative control, and is more effective than each of vorinostat and paclitaxel which demonstrated negligible efficacy. The efficacy of EDO-S101 was determined to be comparable to that of carboplatin. In relation to combination therapies, treatment with EDO-S101 further reduced metabolic activity when used in combination with carboplatin, paclitaxel and carboplatin+paclitaxel, compared to said treatments in the absence of EDO-S101. Combination of EDO-S101+carboplatin was determined to be the most effective combination in reducing cell viability.

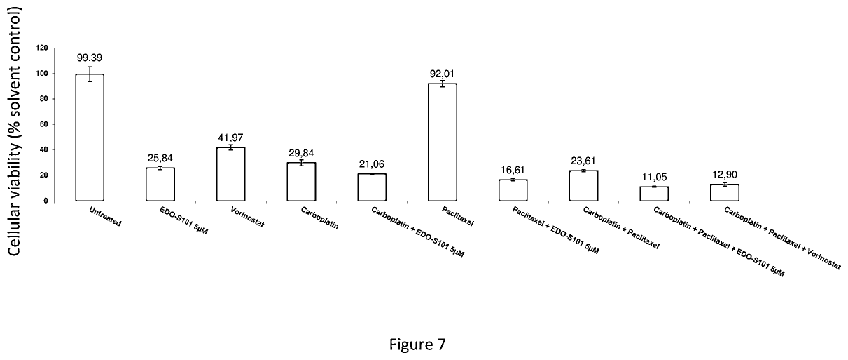

FIG. 7 provides treatment results evaluated with the ATP assay after 72 h using samples from primary ovarian cancer patient #5296. Treatment with EDO-S101 resulted in enhanced reduction in metabolic activity compared to vorinostat, carboplatin or paclitaxel. Furthermore, treatment with EDO-S101 demonstrated enhanced efficacy when in combination with carboplatin, paclitaxel and carboplatin+paclitaxel, compared to said treatments in the absence of EDO-S101. The combination of EDO-S101+carboplatin+paclitaxel resulted in the most pronounced reduction in metabolic activity and was more effective than the combination of carboplatin+paclitaxel+vorinostat.

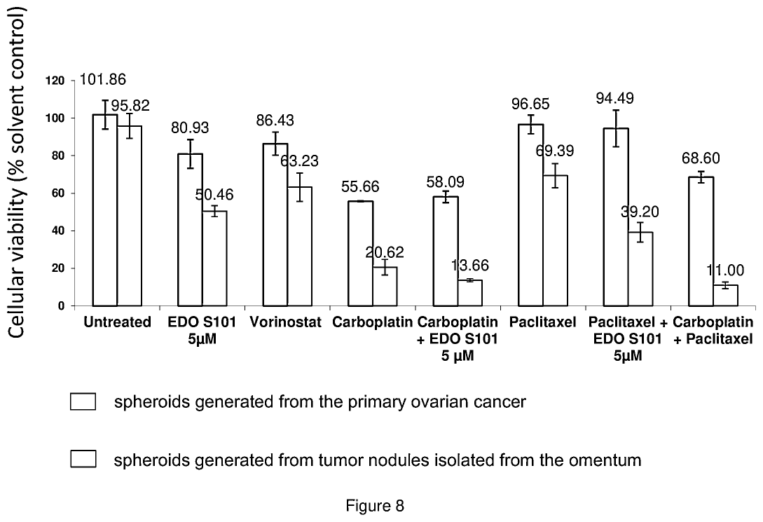

FIG. 8 provides treatment results evaluated with the ATP assay after 72 h using samples from primary ovarian cancer patient #5310 and compares EDO-S101 alone and in combination with carboplatin or paclitaxel in tumour samples taken from the ovary and from the omentum. Treatment of spheroids with a combination of EDO-S101+carboplatin resulted in comparable efficacy to that of carbopatin as a monotherapy or of carboplatin+paclitaxel. The combination of EDO-S101+carboplatin was observed to be more effective in reducing metabolic activity than vorinostat or paclitaxel alone. The data shows that EDO-S101 is effective in treating ovarian cancer from the omentum.

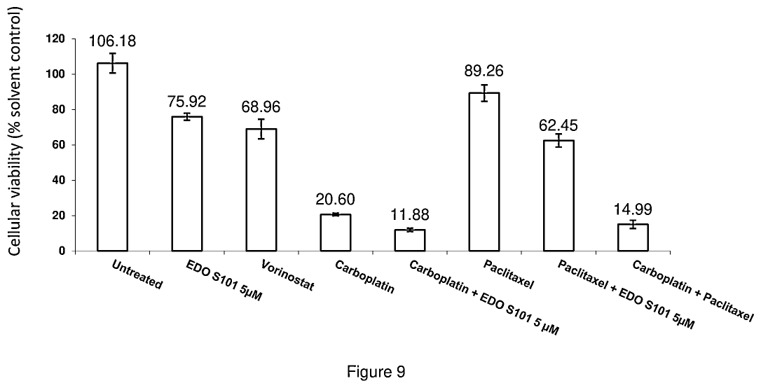

FIG. 9 provides treatment results evaluated with the ATP assay after 72 h using samples from primary ovarian cancer patient #5311. Treatment of spheroids with EDO-S101 resulted in comparable efficacy to that observed in spheroids treated with vorinostat. Treatment with a combination of EDO-S101+carboplatin resulted in the most significant reduction in metabolic activity of all the treatments investigated, including carboplatin as a monotherapy and the combination of carboplatin+paclitaxel. A combination of EDO-S101+paclitaxel was also determined to be more effective than paclitaxel alone.

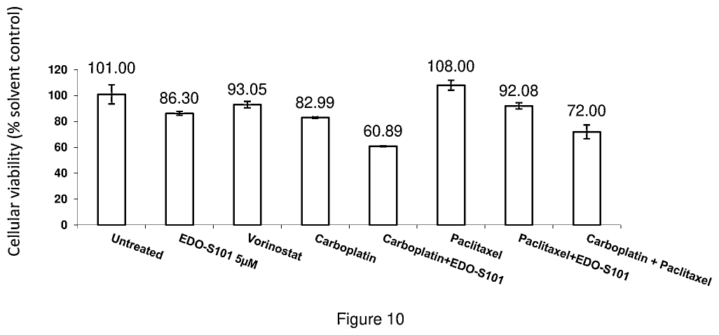

FIG. 10 provides treatment results evaluated with the ATP assay after 72 h using samples from primary ovarian cancer patient #5329. In this patient sample, treatment with EDO-S101 alone resulted in comparable or improved activity compared to vorinostat and carboplatin as monotherapies. The most enhanced reduction in cell metabolic activity was observed with a combination of carboplatin+EDO-S101, compared to all other treatments investigated. As such, the combination of carboplatin+EDO-S101 resulted in the most pronounced reduction in metabolic activity compared to any other agent, either alone or in combination.

### Example 3

A Phase 1/2 Study to Investigate the Safety, Pharmacokinetics and Efficacy of EDO-S101, a First-in-Class Alkylating Histone Deacetylase Inhibition (HDACi) Fusion Molecule, in Patients with Advanced Solid Tumors

### Purpose

Phase 1: To determine the safety, tolerability, maximum tolerated dose (MTD), and recommended phase 2 dose (RP2D) of EDO-S101 as a single agent in patients with solid tumors who have progressed after at least one (1) line of standard therapy.

Phase 2: To evaluate the efficacy of EDO-S101 in selected tumor types.

| Condition | Intervention | Phase |
|---|---|---|
| Phase 1: | Drug: EDO-S101 | Phase 1/2 |
| Advanced or metastatic Solid Tumors | | |
| Phase 2: | | |
| small cell lung cancer (SCLC) | | |
| soft tissue sarcoma or non-Kit GIST | | |
| triple negative breast cancer | | |
| ovarian cancer | | |

Study Type: Interventional
Study Design: Intervention Model: Single Group Assignment
Masking: Open Label
Primary Purpose: Treatment

### Primary Outcome Measures:

### Phase 1: Dose Escalation Until Maximum Administered Dose (MAD):

Determine Maximum Tolerated Dose at optimal infusion schedule

### Phase 2: Evaluation of Toxicity and Response Rate in Selected Solid Tumor Cohorts:

Confirm recommended Phase 2 dose and optimal infusion schedule in select solid tumors
Determine objective response rate and clinical benefit rate in selected solid tumors

### Secondary Outcome Measures:

### Phase 1: Dose Escalation Until MAD:

Determine maximum plasma concentration (Cmax) of EDO-S101

### Phase 2: Evaluation of Toxicity and Response Rate in Selected Solid Tumor Cohorts:

To evaluate safety and tolerability of the RP2D of the selected schedule of EDOS101.
To determine the progression free survival time for patients who received the RP2D at the optimal infusion schedule
To determine overall survival for patients who received the RP2D at the selected study drug administration schedule.
To establish the trough PK profiles of EDO-S101.
Estimated Enrollment: 158

### Phase 1:

Schedule A: EDO-S101, IV, 60 mg/m² up to 150 mg/m² Day 1 and 15 of each 28 day cycle
Schedule B: EDO-S101, IV, 60 mg/m² up to 150 mg/m² Day 1, 8 and 15 of each 28 day cycle

### Phase 2:

The RP2D and selected schedule will be further investigated in patients with specific types of solid tumors: relapsed/refractory SCLC, soft tissue sarcoma, non-Kit GIST, triple negative breast, and ovarian cancers.

### Detailed Description

EDO-S101 I entity, a first-in-class fusion molecule of an alkylator, bendamustine and a histone-deacetylase inhibitor (HDACi), vorinostat. This phase 1/2 study will enroll patients with solid tumours. This phase 1/2 study will enroll patients with various advanced solid tumours.

### The Study Consists of 2 Phases:

Phase 1: Dose Escalation until MAD
Phase 2: Evaluation of Toxicity and Response Rate in Selected Solid Tumor Cohorts

The study is designed as an open label, Phase 1/2 trial of single agent EDOS101. The phase 1 portion of the study is designed to define the MTD for two (2) administration schedules by evaluating toxicities during dose escalation until MAD. The phase 2 portion of the study is designed to evaluate ORR and CBR at four (4) or six (6) months depending on the type of solid tumor.

### Eligibility

Ages Eligible for Study: 18 Years and older (Adult, Senior)
Sexes Eligible for Study: All
Accepts Healthy Volunteers: No

### Criteria

### Inclusion Criteria for Phase 1 and Phase 2 Portions of Study:

1. Signed informed consent.
2. patients ≥18 years at signing the informed consent.
3. Diagnosis of advanced or metastatic solid tumors, disease should have progressed following at least one line of standard therapy.
4. Patients with secondary metastasis to the CNS are eligible if they have met certain criteria.
5. Evaluable disease; either measurable on imaging or with informative tumor marker.
6. Discontinuation of previous cancer therapies at least three (3) weeks or 5 half-lives, whichever is shorter.
7. Eastern Cooperative Oncology Group (ECOG) performance status≤2
8. Neutrophils ≥1,500 μL.
9. Platelets ≥100,000 μL.
10 Aspartate aminotransferase/alanine aminotransferase (AST/ALT)≤3 upper limit of normal (ULN). In cases with liver involvement ALT/AST≤5×ULN.
11. Total bilirubin≤1.5 mg/dl unless elevated due to known Gilbert's syndrome.
12. Creatinine≤1.5 ULN.
13. Serum potassium within normal range.
14. If female of child-bearing potential (i.e. not postmenopausal or surgically sterile), must be willing to abstain from sexual intercourse or employ an effective barrier or medical method of contraception during the study drug administration and follow-up periods. If male, must be sterile or willing to abstain from sexual intercourse or employ a barrier method of contraception during the study treatment and at least 6 months following last treatment.

### Exclusion Criteria for Phase 1 and Phase 2 Portions of Study:

1. Patients with primary central nervous system (CNS) cancer.
2. Patients with QTc interval >450 msec for male and >470 msec for female.
3. Patients who are on treatment with drugs known to prolong the QT/QTc interval.
4. Patients who are on treatment with Valproic Acid in any of its indication (epilepsy, mood disorder) must be excluded or must stop using the medication.
5. Any serious medical condition that interferes with adherence to study procedures.
6. Prior history of solid tumor malignancy diagnosed within the last three (3) years of study enrollment excluding adequately treated basal cell carcinoma of the skin, squamous cell carcinoma of the skin, or in situ cervical cancer, in situ breast cancer, in situ prostate cancer (patients must have shown no evidence of active disease for 2 years prior to enrollment)
7. Pregnant or breast feeding females.
8. New York Heart Association (NYHA) stage III/IV congestive heart failure, arrhythmias not adequately controlled, or other significant co-morbidities [e.g. active infection requiring systemic therapy, history of human immunodeficiency virus (HIV) infection, or active Hepatitis B or Hepatitis C].
9. Use of other investigational agents within 30 days or 5 half-lives prior to the first dose of study drug. As long as patient has recovered from any related toxicities ≥Grade 1.
10. Steroid treatment within seven (7) days prior to study treatment. Patients that require intermittent use of bronchodilators, topical steroids or local steroid injections will not be excluded from the study. Patients who have been stabilized to 10 mg orally prednisolone PO QD (or equivalent), daily or less seven (7) days prior to study drug administration are allowed.

### Phase 2 Tumor-Specific Eligibility Criteria

Phase 2 patients must meet the cohort-specific inclusion/exclusion criteria in addition to the general inclusion/exclusion criteria for Phase 1 and Phase 2 study listed above.

### Cohort 1 Patient Population: Relapsed/Refractory SCLC

1. Histologically or cytological confirmed limited or extensive disease stage of SCLC. The disease should be progressing during or relapsing after the previous treatment.
2. At least one line of prior combination chemotherapy including adequate doses of platinum compound and having progressed during therapy or after the previous treatment.
3. At least 3 weeks or 5 half-lives, whichever is shorter, should have elapsed since prior treatment as long as the patient recovered from any related toxicities to ≤ Grade 1.
4. Prior radiotherapy is acceptable provided the patient has recovered from any radiotherapy related acute toxicities.

### Cohort 2 Patient Population: Relapsed/Refractory Soft Tissue Sarcoma or Non-Kit GIST

1. Histologically confirmed diagnosis of advanced, unresectable, or metastatic soft tissue sarcoma not amenable to curative treatment with surgery or radiotherapy excluding: chondrosarcoma, neuroblastoma, osteosarcoma, embryonal rhabdomyosarcoma, or Kaposi sarcoma.
2. Must have received at least one prior first line combination chemotherapy regimen or at least two first line single-agent regimens. Adjuvant chemotherapy not considered first line, unless disease progression within 6 months of treatment.
3. The disease should be progressing/relapsed during or after the previous treatment. At least 3 weeks should have elapsed since prior chemotherapy or 5 half-lives, whichever is shorter, as long as the patient recovered from any related toxicities to ≤Grade 1.
4. Presence of measurable disease as defined by the Response Evaluation Criteria in Solid Tumors (RECIST 1.1, Eisenhauer et al. 2009).

### Cohort 3 Patient Population: Relapsed/Refractory Triple Negative Breast Cancer

1. Histologically or cytologically confirmed locally advanced or metastatic Triple Negative Metastatic Breast Cancer.
2. Must have received at least one line of chemotherapy, at least 3 weeks should have relapsed since prior chemotherapy or 5 half-lives, whichever is shorter, as long as the patient recovered from acute toxicity of previous therapies to ≤grade 1.
3. Prior radiotherapy is acceptable provided it was applied within 4 four weeks before starting of this trial and the patient recovered from any radiotherapy related acute toxicities.
4. The disease should be progressing/relapsed during or after the previous treatment.
5. Presence of measurable disease as defined by the Response Evaluation Criteria.

### Cohort 4 Patient Population: Relapsed/Refractory Ovarian Cancer

1. Histologically or cytologically confirmed advanced ovarian cancer: epithelial ovarian cancer, primary peritoneal cancer or fallopian tube cancer (excluding borderline ovarian cancer) that is resistant or refractory to platinum therapy.
   a. Platinum-resistant ovarian cancer is defined as disease that responded to primary platinum therapy and then progressed within 6 months or disease that progressed during or within six months of completing a subsequent platinum therapy.
   b. Primary platinum refractory disease is defined as disease that has not responded to a platinum-based regimen or experienced disease recurrence within 3 months of completing a first-line platinum-based regimen.
2. The disease should be progressing/relapsed during or after the previous treatment. At least 3 weeks should have elapsed since prior chemotherapy or 5 half-lives, whichever is shorter, as long as the patient recovered from acute toxicity of previous therapies to ≤grade 1.
3. Presence of measurable disease as defined by the Response Evaluation Criteria in Solid tumors (RECIST 1.1, Eisenhauer et al. 2009).

1. Tinostamustine or a pharmaceutically acceptable salt thereof for use in the treatment of ovarian cancer in a patient in need thereof.

2. Tinostamustine or a pharmaceutically acceptable salt thereof for use in the treatment of ovarian cancer according to claim 1 wherein the ovarian cancer is from the omentum.

3. Tinostamustine or a pharmaceutically acceptable salt thereof for use in the treatment of ovarian cancer according to any preceding claim, wherein the cancer is platinum resistant.

4. Tinostamustine or a pharmaceutically acceptable salt thereof for use in the treatment of ovarian cancer according to any preceding claim, wherein the cancer is BRCA-1/2 wildtype.

5. Tinostamustine or a pharmaceutically acceptable salt thereof for use in the treatment of ovarian cancer according to any preceding claim, wherein the cancer is relapsed and/or refractory.

6. Tinostamustine or a pharmaceutically acceptable salt thereof for use in the treatment of ovarian cancer according to any preceding claim, wherein the cancer is a primary cancer.

7. Tinostamustine or a pharmaceutically acceptable salt thereof for use in the treatment of ovarian cancer according to any preceding claim, wherein tinostamustine or a pharmaceutically acceptable salt thereof is used in combination with one or more further agent(s).

8. Tinostamustine or a pharmaceutically acceptable salt thereof for use in the treatment of ovarian cancer according to claim 7, wherein the further compounds are selected from carboplatin and/or paclitaxel.

9. Tinostamustine or a pharmaceutically acceptable salt thereof for use in the treatment of ovarian cancer according to any one of claims 7 to 8, wherein tinostamustine and further agent(s) are administered concurrently, sequentially or separately.

10. Tinostamustine or a pharmaceutically acceptable salt thereof for use in the treatment of ovarian cancer according to any one of claims 1 to 6, wherein the tinostamustine or a pharmaceutically acceptable salt thereof is used as a monotherapy.

11. Tinostamustine or a pharmaceutically acceptable salt thereof for use in the treatment of ovarian cancer according to any preceding claim, wherein tinostamustine or a pharmaceutically acceptable salt thereof is administered intravenously to the patient in need thereof at a dosage level of from 0.3 mg/m² to 300 mg/m² body surface area of the patient; or from 60 to 150 mg/m² body surface area of the patient; or from 80 to 100 mg/m² body surface area of the patient.

12. Tinostamustine or a pharmaceutically acceptable salt thereof for use in the treatment of ovarian cancer according to any one of claims 1 to 11, wherein tinostamustine or a pharmaceutically acceptable salt thereof is administered intravenously to the patient in need thereof on days 1, 8 and 15 of a 28 day treatment cycle; or on days 1 and 15 of a 28 day treatment cycle.

13. Tinostamustine or a pharmaceutically acceptable salt thereof for use in the treatment of ovarian cancer according to any one of claims 1 to 12, wherein tinostamustine or a pharmaceutically acceptable salt thereof is administered intravenously to the patient in need thereof over an infusion time of 60 minutes; or an infusion time of 45 minutes; or an infusion time of 30 minutes.

14. Tinostamustine or a pharmaceutically acceptable salt thereof for use in the treatment of ovarian cancer according to any one of claims 1 to 13, wherein tinostamustine or a pharmaceutically acceptable salt thereof is administered intravenously to the patient in need thereof at a dosage level of from 80 mg/m² to 100 mg/m² body surface area of the patient, on days 1 and 15 of a 28 day treatment cycle, over an infusion time of 60 minutes.

15. Tinostamustine or a pharmaceutically acceptable salt thereof for use in the treatment of ovarian cancer according to any one of claims 1 to 14, wherein the patient is treated with tinostamustine or a pharmaceutically acceptable salt thereof and radiotherapy.

16. Tinostamustine or a pharmaceutically acceptable salt thereof for use in the treatment of ovarian cancer according to claim 15, wherein said radiotherapy treatment is given to the patient in need thereof at a dose of 1 to 5 Gy over 5-10 consecutive days, and preferably 2 Gy over 5-10 consecutive days.

17. A combination comprising tinostamustine or a pharmaceutically acceptable salt thereof, and further agent(s) selected from carboplatin and/or paclitaxel.

18. The combination according to claim 17, wherein the molar ratio of compound of formula I and further agent(s) are selected from: 1:2000 to 2000:1; 1:100 to 1:2000; 1:500 to 1:1000; 1:500 to 1:900.

19. A pharmaceutical composition comprising a pharmaceutically acceptable carrier and a combination according to any one of claims 17 to 18.

20. A kit comprising tinostamustine or a pharmaceutically acceptable salt thereof with instructions for treating ovarian cancer; or a kit comprising tinostamustine or a pharmaceutically acceptable salt thereof, and further agent(s) selected from carboplatin and/or paclitaxel, optionally with instructions for treating ovarian cancer.

21. The combination according to any one of claims 17 to 18, composition according to claim 19 or kit according to claim 20, for use as a medicament.

22. The combination according to any one of claims 17 to 18, composition according to claim 19 or kit according to claim 20, for use in the treatment of cancer in a patient in need thereof.

23. The combination, composition or kit according to claim 22 wherein the cancer is ovarian cancer.
